(19) United States
(12) Patent Application Publication
(10) Pub. No.: US 2021/0382521 A1
(43) Pub. Date: Dec. 9, 2021

(54) CASE FOR A MOBILE DEVICE AND A MOBILE DEVICE

(71) Applicant: Adebowale Ajagbe, Livonia, MI (US)

(72) Inventor: Adebowale Ajagbe, Livonia, MI (US)

(21) Appl. No.: 16/910,853

(22) Filed: Jun. 24, 2020

Related U.S. Application Data
(60) Provisional application No. 63/035,665, filed on Jun. 5, 2020.

(51) Int. Cl. G06F 1/16; A61L 9/20; A61L 9/14

(52) U.S. Cl. CPC G06F 1/1607; A61L 9/20; A61L 9/14; A61L 2202/16; A61L 2209/12; A61L 2209/133; A61L 2202/11; G06F 1/1656

(57) ABSTRACT

A case for a mobile device includes a housing having an open top, the housing configured to receive the mobile device and a first light emitting device disposed within the housing. A light guide is optically connected to the first light emitting device. The light guide is configured to direct light over an exposed surface of the mobile device. The case may include a first and second cartridge for dispensing an antiseptic and fragrance, respectively.

### CROSS-REFERENCE TO RELATED APPLICATION

This application claims priority to U.S. Provisional Application No. 63/035,665 filed on Jun. 5, 2020, the contents of which are incorporated herein by reference.

### TECHNICAL FIELD

The disclosure relates to a case for a mobile device and a mobile device.

### BACKGROUND

Mobile devices are constantly handled. As such, contaminants such as bacteria and virus may be left on the surface of the mobile device. It is known to use a disinfectant wipe or spray is to kill the contaminants. This requires the user to carry such a disinfectant.

Accordingly, it remains desirable to have a case for a mobile device having a means for disinfecting the mobile device.

### SUMMARY

One aspect of the disclosure is a case for a mobile device. The case includes a housing having an open top. The housing is configured to receive the mobile device. The case further includes a first light emitting device. The first light emitting device is disposed within the housing. The case further includes a light guide. The light guide is optically connected to the first light emitting device. The light guide is configured to direct light over an exposed surface of the mobile device.

In one aspect, the case includes a battery configured to power the first light emitting device. The battery may be removably attached to the case or integrally formed thereto. The case may further include an actuator configured to actuate the first light emitting device.

In another aspect, the first light emitting device is configured to emit ultraviolet light. The first light emitting device may be integrally formed to the housing, and the first light guide may be disposed along a first peripheral edge of the housing. The first light emitting device may be further configured to emit visible light.

In yet another aspect, the light guide is disposed along a first peripheral edge and a second peripheral edge of the housing, the second peripheral edge being opposite of the first peripheral edge. Accordingly, an ultraviolet light may be directed to the exposed surface of the mobile device from each side.

In yet another aspect, the case includes a second emitting device. The second emitting device is disposed on a back surface of the housing so as to be opposite of the open top. The second light emitting device may be fixed or removably attached to the back surface of the housing. In instances where the second light emitting device is removably attached, a wire electrically couples the second light emitting device to the battery so as to power the second light emitting device when the second light emitting device is removed from the housing. In such an aspect, the second light emitting device may be used as a wand to disinfect various surfaces.

In yet another aspect, the case includes a first cartridge configured to hold a fluid. The housing includes a compartment configured to receive the cartridge. The case further includes a dispensing mechanism fluidly coupled to the first cartridge when the first cartridge is disposed within the compartment so as to dispense the fluid through a port disposed on a back surface of the housing.

In yet another aspect, the case includes a slot and a second cartridge. The slot is disposed along a peripheral side of the housing. The second cartridge is configured to emit a scent and at least one hole disposed on the back surface of the housing, the at least one hole in fluid communication with the slot.

Accordingly, a case having an integrated disinfectant is provided wherein the exposed surface of the mobile device may be disinfected with ultraviolet light. Further, the case provides a means for supplying an antiseptic to the user. Further, the case provides a means for releasing a fragrance.

In yet another aspect, a case includes a housing for housing an electronic inhaling device such as a device commonly referenced as a vape pen or a Juul e-cigarette. The case may further include an adaptor, the adaptor configured to electrically couple the electronic inhaling device to a battery of the case.

In yet another aspect, the first cartridge is removably attached to a back surface of the case. The first cartridge may be configured to dispense a fluid such as an antiseptic or may be configured to hold items such as cosmetic items.

In yet another aspect, a mobile device is provided. The mobile device includes a first light emitting device disposed within the housing. A light guide is optically connected to the first light emitting device. The light guide is configured to direct light over the exposed surface of the mobile device. The light guide may be disposed along a first peripheral edge and a second peripheral edge of the housing, the second peripheral edge being opposite of the first peripheral edge.

In yet another aspect of the mobile device, the mobile device further includes a second emitting device disposed on a back wall of the housing.

In yet another aspect of the mobile device, the mobile device includes a compartment and a first cartridge. The first cartridge is configured to hold a fluid and the compartment is disposed within the housing and is configured to receive the cartridge. A dispensing mechanism may be fluidly coupled to the first cartridge when the first cartridge is disposed within the compartment so as to dispense the fluid through a port disposed on a back surface of the housing.

In another aspect of the mobile device, the mobile device includes a second cartridge and a slot. The slot is disposed along a peripheral side of the housing. The second cartridge is configured to emit a scent. The housing includes a hole. The hole is disposed on the back wall of the housing and is in fluid communication with the slot.

### BRIEF DESCRIPTION OF THE DRAWINGS

The embodiments set forth in the drawings are illustrative and exemplary in nature and not intended to limit the subject matter defined herein. The following description of the illustrative embodiments can be understood when read in conjunction with the following drawings, where like structure is indicated with like reference numerals and in which:

FIG. 1 is a perspective view illustratively depicting a case, taken from the back, according to a first embodiment described herein.

FIG. 2 is a view of FIG. 1 taken from the front.

FIG. 3 is a partial cross-sectional view of the case shown in FIG. 1 with a mobile device inserted into the opening.

FIG. 4 is a top down view of a case taken from the front according to a second embodiment.

FIG. 5 is a perspective view of a case taken from the back according to a third embodiment.

FIG. 6 is a perspective view of a case taken from the back according to a fourth embodiment.

FIG. 7 is a perspective view of an adaptor according to a first aspect.

FIG. 8 is a perspective view of an adaptor according to a second aspect.

FIG. 9 is a perspective view of a case for use with the adaptor shown in FIGS. 7 and 8.

FIG. 10 is a perspective view of a case according to another aspect.

FIG. 11 is a view of a back wall of a mobile device according to one aspect described herein.

FIG. 12 is a view of the exposed surface of the mobile device shown in FIG. 11.

### DETAILED DESCRIPTION OF PREFERRED EMBODIMENTS

A case for a mobile device includes a housing having an open top. The housing is configured to receive the mobile device. The case further includes a first light emitting device. The first light emitting device is disposed within the housing. The case further includes a light guide. The light guide is optically connected to the first light emitting device. The light guide is configured to direct light over an exposed surface of the mobile device. Preferably the first light emitting device is configured to emit ultraviolet light. Accordingly, the case is configured to disinfect the exposed surface of the mobile device with ultraviolet light.

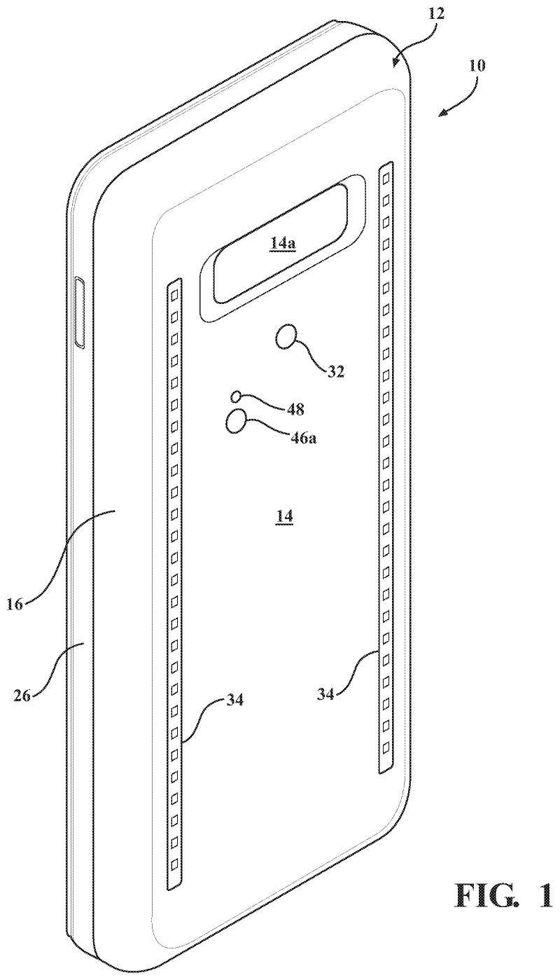

With reference now to FIG. 1, a first embodiment of a case 10 is provided. The case 10 includes a housing 12. The housing 12 may be formed of a generally rigid material suitable for an injection molding process, illustratively including a polypropylene. The housing 12 includes a base 14 and a peripheral wall 16 bounds the base 14 so as to define an open top 18. The base 14 is a generally planar member. The base 14 includes an opening 14a for accommodating a lens assembly of the mobile device 100. It should be appreciated that the shape of the housing 12 is provided for illustrative purposes and is not limiting to the scope of the appended claims. For illustrative purposes, the housing 12 is dimensioned to accommodate an iPhone®; however, it should be appreciated that the housing 12 may be dimensioned to accommodate other types of mobile device 100s, such as a mobile device 100 manufactured by Apple, Samsung, Google or the like.

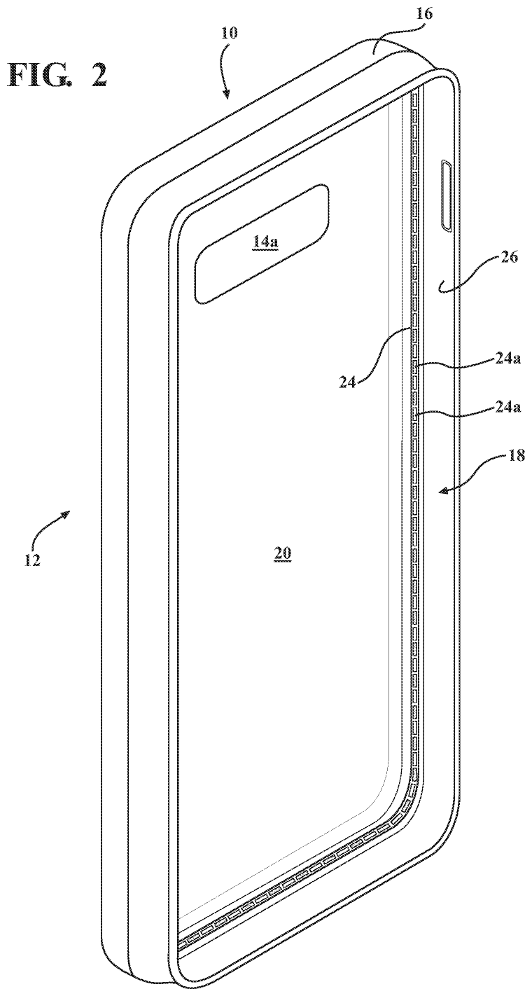

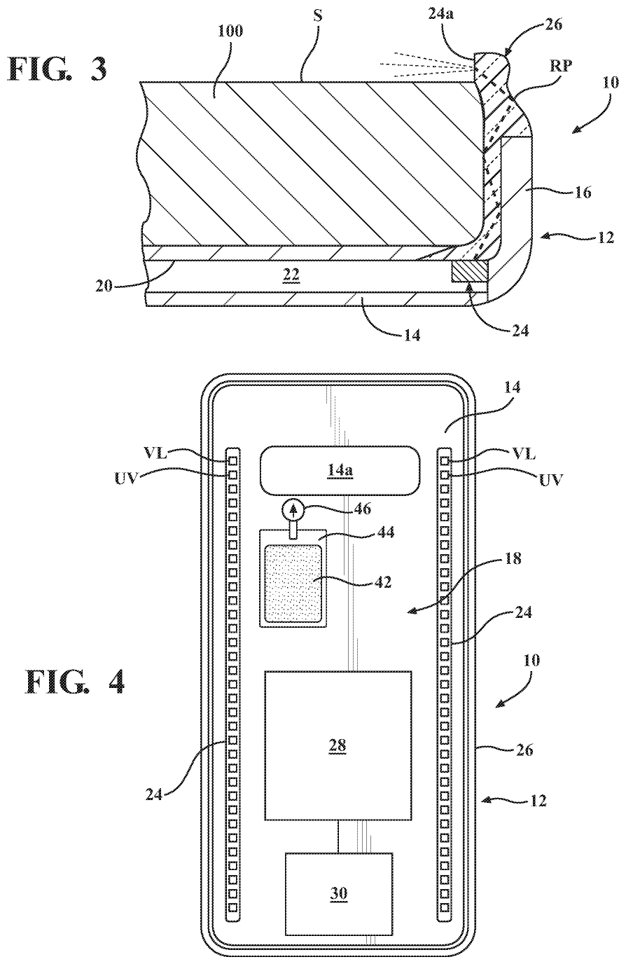

With reference now to FIGS. 2 and 3, a perspective view of the front of the case 10 is provided. The front of the case 10 includes a front wall 20. The front wall 20 is spaced apart from the base 14 so as to define an interior space 22 for housing various components as described further below. The case 10 further includes a first light emitting device 24. The first light emitting device 24 is disposed within the interior space 22 of the housing 12. The first light emitting device 24 is illustratively shown as a plurality of light sources 24a, such as a light emitting diode, arranged in series with each other so as to extend along the entire peripheral edge of the housing 12. However, the lights may be arranged along an axis. In such an embodiment, the case 10 may include a pair of first light emitting devices 24, each extending along an axis which is generally the length of the housing 12 and on opposite sides of the housing 12 so as to be on a first peripheral edge and a second peripheral edge. The first peripheral edge and the second peripheral edge being opposite of each other.

With reference again to FIGS. 1 and 2 and now also to FIG. 3, the case 10 further includes a light guide 26. The light guide 26 is optically connected to the first light emitting device 24. The light guide 26 is configured to direct light over an exposed surface of the mobile device 100. The light guide 26 may be formed of a material configured to guide light. Any such material currently known or later developed may be adapted for use herein, illustratively including a synthetic resin film material configured to allow light to flow through, an elastomer material such as an ultra-thin elastomer, a polyurethane, and a high optical grade material or, alternatively, a low optical grade material.

FIG. 3 shows a mobile device 100 disposed within the housing 12. The first light emitting device 24 is optically coupled to the light guide 26. FIG. 3 shows an example of the ultraviolet light propagating through the light guide 26 so as to shine onto the exposed surface “S” of the mobile device 100. The light guide 26 is shown as having a reflective point “RP” which reflects the light through a side wall 26a of the light guide 26, shining ultraviolet light onto the exposed surface “S” of the mobile device 100.

With reference now to FIG. 4, a top down view of the case 10 with the front wall 20 removed. In one aspect, the case 10 includes a battery 28 configured to power the first light emitting device 24. An electronic circuit 30 may be provided to actuate the first light emitting device 24 in accordance with a predetermined protocol. The electronic circuit 30 may be a computer program product including a non-transitory, machine-readable medium storing instructions which, when executed by a programmable processor, causes programmable processor to perform the predetermined protocol. For instance, the electronic circuit 30 may be programmed to change the intensity of the ultraviolet light, provide a pulsing feature, an automatic shut off, a duration and the like. The battery 28 may be removably attached to the case 10 or integrally formed thereto.

It should be appreciated that the first light emitting device 24 may be configured to emit visible light as well as ultraviolet light. For example, the first light emitting device 24 may include a plurality of light emitting diodes (LEDs) and some of the LEDs may be configured to transmit visible light while others are configured to transmit ultraviolet light. In one aspect, the first light emitting device 24 has the LEDs alternating between a visible light LED “VL” and an ultraviolet light LED “UV”. FIG. 4 depicts an embodiment where the case 10 includes a pair of first light emitting devices 24. The first light emitting devices 24 may be integrally formed to the housing 12. The first light guide 26 are shown disposed along a respective first and second peripheral edge of the housing 12.

With reference again to FIG. 1, the case 10 may further include an actuator 32 configured to actuate the first light emitting device 24. For illustrative purposes, the actuator 32 is shown as a button. FIG. 1 further discloses another aspect of the case 10 wherein the case 10 includes a second light emitting device 34. The second light emitting device 34 is disposed on a back surface of the housing 12 so as to be opposite of the open top 18. The second light emitting device 34 may be fixed or removably attached to the back surface of the housing 12.

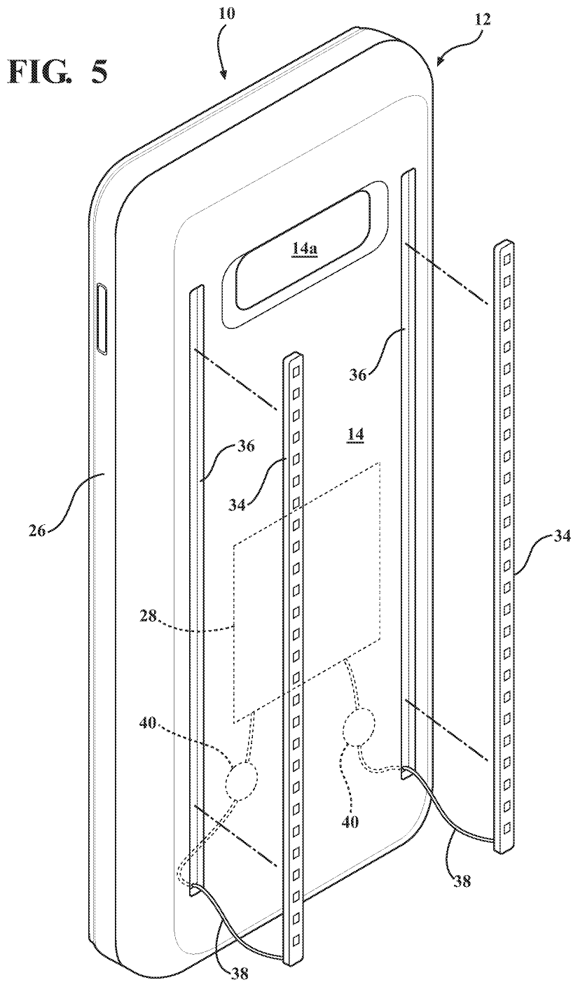

With reference now to FIG. 5, a depiction of a case 10 where the second light emitting device 34 is removably attached is provided. The case 10 includes a light device housing 36 disposed on a back surface of the base 14. The light device housing 36 is configured to fittingly receive the second light emitting device 34. Preferably, the light device housing 36 has a depth configured to retain the outer surface of the second light emitting device 34 flush with the back surface of the base 14 of the housing 12.

The case 10 further includes a wire 38 electrically coupling the second light emitting device 34 to the battery 28 so as to power the second light emitting device 34 when the second light emitting device 34 is removed from the housing 12. The case 10 may include a spooling mechanism 40 configured to spool the wire 38 so as to allow the second light emitting device 34 to extend from the case 10 and to retract and spool the wire 38 when the second light emitting device 34 is seated in the light device housing 36. In such an aspect, the second light emitting device 34 may be used as a wand to disinfect various surfaces.

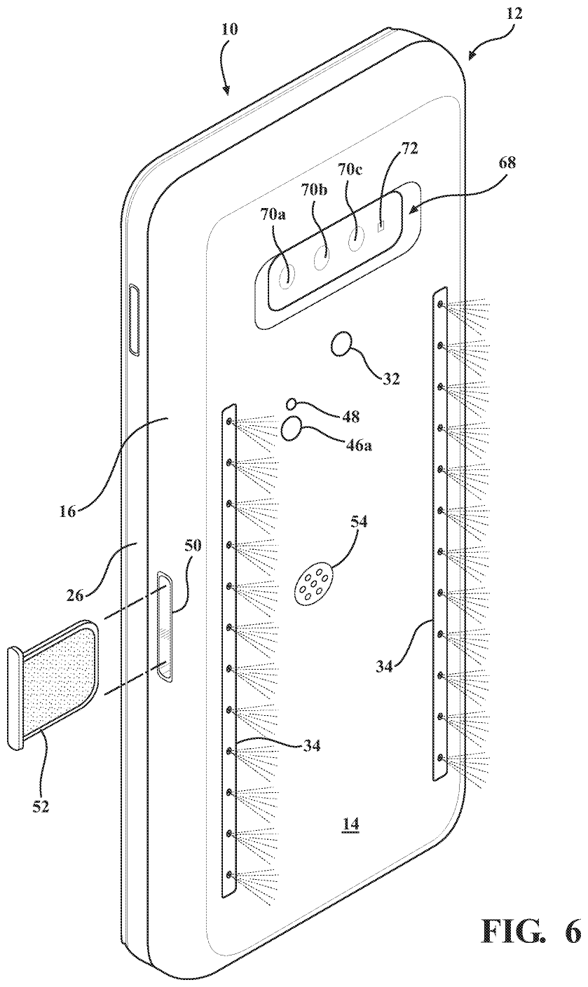

With reference again to FIG. 4, another aspect of the case 10 is provided. The case 10 may include a first cartridge 42 configured to hold a fluid. In particular, the first cartridge 42 is configured to hold an antiseptic. The housing 12 includes a compartment 44 configured to receive the first cartridge 42. The case 10 further includes a dispensing mechanism 46 fluidly coupled to the first cartridge 42 when the first cartridge 42 is disposed within the compartment 44 so as to dispense the fluid through a port 48 disposed on a back surface of the housing 12. The port 48 is shown in FIGS. 1 and 6. For illustrative purposes, the dispensing mechanism 46 may be a pump which is actuated by pressing a button 46a disposed on a back of the base 14.

With reference now to FIG. 6 another aspect of the case 10 is provided wherein the case 10 is configured to emit a fragrance. The case 10 includes a slot 50 disposed along the peripheral wall 16 of the housing 12. The case 10 further includes a second cartridge 52 configured to emit a scent and at least one hole 54 disposed on the back surface of the housing 12, the at least one hole 54 in fluid communication with the slot 50. Accordingly, the second cartridge 52 is seated within the slot 50 and the hole 54 is preferably located above the second cartridge 52 so as to allow fragrance from the second cartridge 52 to diffuse freely through the holes 54.

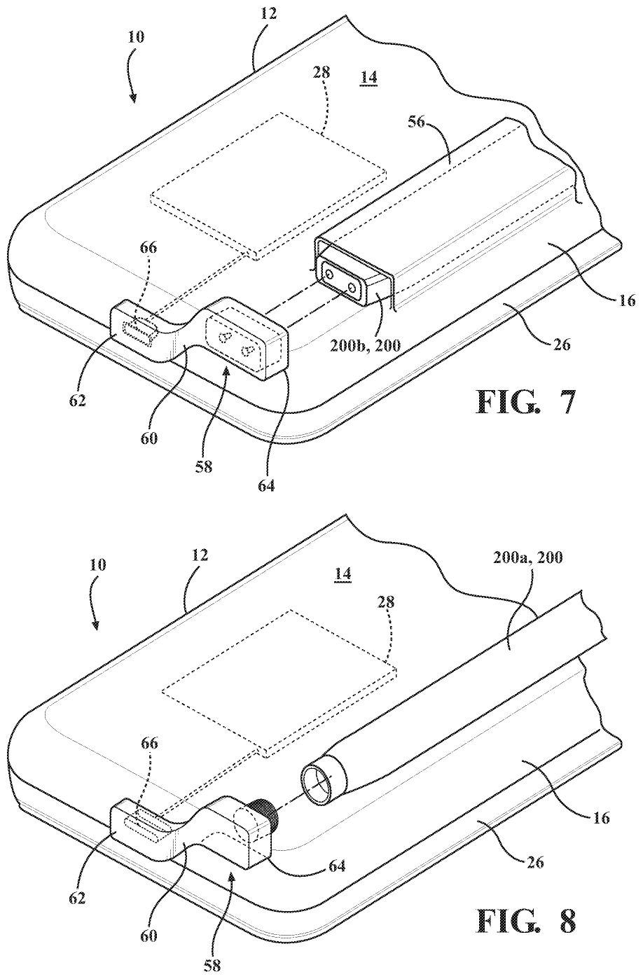

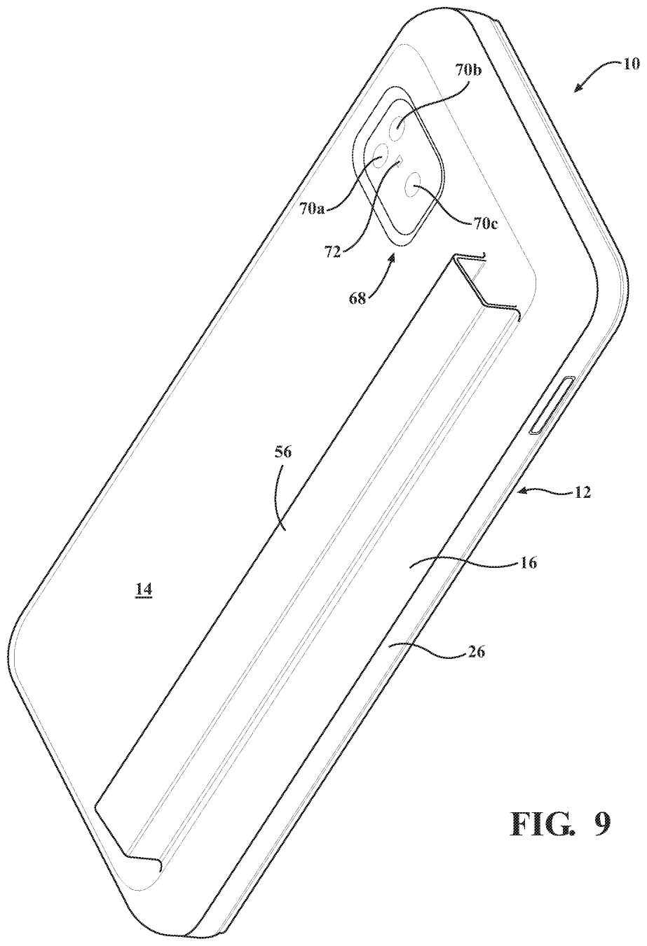

With reference now to FIGS. 7-9, the case 10 may further include an exterior housing 56 for housing 12 an electronic inhaling device 200 such as a device commonly referenced as a vape pen 200a or a Juul e-cigarette 200b. FIG. 9 provides an example of an exterior housing 56 which is configured to house a Juul e-cigarette 200b. However, it should be appreciated that the exterior housing 56 may be shaped differently depending on the type of electronic inhaling device 200 that the case 10 is be used with. The exterior housing 56 is an elongated member having a pair of open ends.

The case 10 may further include an adaptor 58 configured to be removable attached to the case 10. The adaptor 58 includes a body 60 having a first mating portion 62 and a second mating portion 64. The first mating portion 62 is offset from the second mating portion 64. The first mating portion 62 is configured to couple with an input 66 of the case 10. The input 66 is coupled to the battery 28. The input 66 may any commercial input currently known or later developed. For example, the input 66 may be a port configured to receive a USB-C connector or a connector that is compatible with an iPhone®, commonly known as a lightning connector.

FIG. 7 shows an adaptor 58 having a first mating portion 62 configured to engage an input 66 of the case 10, wherein the input 66 is configured to receive a lightning connector. The second mating portion 64 is offset so as to sit above the base 14 of the housing 12 and is configured to engage an input 66 of a Juul e-cigarette 200b. As shown in FIG. 9, the exterior housing 56 is configured to position the input of the Juul e-cigarette 200b such that the adaptor 58 can electrically connect the battery 28 of the case 10 to the Juul e-cigarette 200b when the Juul e-cigarette 200b is seated within the exterior housing 56.

FIG. 8 shows an adaptor 58 having a first mating portion 62 configured to engage an input 66 of the case 10, wherein the input 66 is configured to receive a USB-C connector. The second mating portion 64 is offset so as to sit above the base 14 of the housing 12 and is configured to engage an input 66 of a Vape pen 200a. Naturally, the exterior housing 56 shown in FIG. 9 may be modified to accommodate a Vape pen 200a and may be configured to position the input of the Vape pen 200a such that the adaptor 58 can electrically connect the battery 28 of the case 10 to the Vape pen 200a when the Vape pen 200a is seated within the exterior housing 56.

In another aspect, the adaptor 58 may be integrally formed to the case 10. In such an embodiment, the first mating portion 62 is removed and the second mating portion 64 is wired directly to the battery 28. Further, the second mating portion 64 may be integrally formed to a bottom end of the exterior housing 56. Accordingly, the case 10 shown in FIGS. 7-9 is adapted to charge an electronic inhaling device 200.

With reference again to FIG. 6, the case 10 may further include a lens assembly 68 having a plurality of lenses 70a, 70b, 70c. The lenses 70a, 70b, 70c may be configured to enhance the image captured by the camera of the mobile device 100. The lenses 70a, 70b, 70c are shown as being aligned along an axis, but it should be appreciated that the lenses 70a, 70b, 70c are configured to cover the lenses of the mobile device 100. For instance, FIG. 9 provides an example of a lens assembly 68 configured to cover the lenses of an iPhone®.

One of the lenses, 70a for example, may be configured to magnify the camera image, whereas another lens, 70b for example, is configured to capture night vision, any such lens currently known and used may be modified and adapted for use herein, illustratively including the a lens having a photocathode. The lens assembly 68 may further include an IR source 72 configured to transmit infrared light, as an example near infrared, so as to further enhance the night vision capabilities of the mobile device 100.

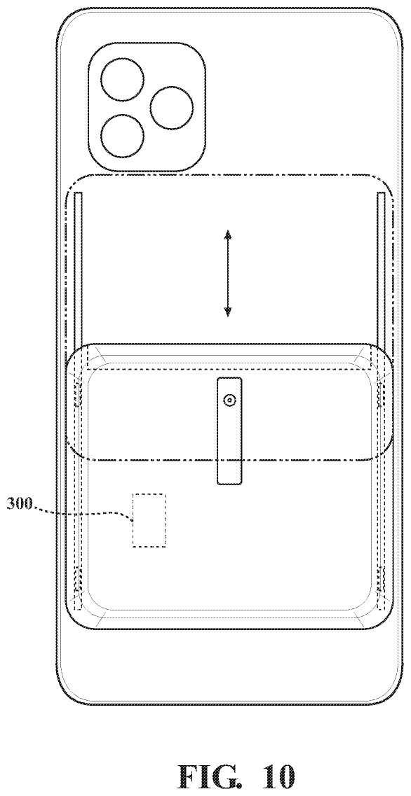

With reference now to FIG. 10, another aspect of case 10 is provided. The housing 12 includes an open top 18 configured to receive the mobile device 100. The first cartridge 42 removably attached to a back surface 14a of the housing 12. In particular, the case 10 includes a means for removably attaching the first cartridge 42 to the back surface 14a of the housing 12. Any such means currently known or later developed may be adapted for use herein, illustratively including a groove 74 disposed on an exterior surface of a base of the housing and a tab 76 disposed on the first cartridge 42. The tab 76 is configured to slide within the groove 74 so as to secure the first cartridge 42 to the housing 12. It should be appreciated that the tab 76 and the groove 74 may be dimensioned to form a locking engagement. As an example, the groove 74 may narrow allowing a pinch-fit engagement with the tab 76 so as to secure the first cartridge 42 to the case 10.

The first cartridge is configured to hold content such as a fluid or cosmetic items such as lip stick, eye-liner, cleaning wipes or the like. For instance, the first cartridge 42 hold an antiseptic. In such an aspect, the first cartridge 42 may further include a dispensing mechanism 46 configured to dispense the antiseptic.

In aspects where the first cartridge 42 is configured to hold a cosmetic item 300, the first cartridge 42 includes a cover 78 configured to open or close the space 80 so as to provide access or contain the cosmetic item stored within the space 80. The cover 78 is indicated in dashed lines and may be hingedly attached to the top of the first cartridge 42. Accordingly, the case 10 may be adapted to hold various items for the user's convenience, such as antiseptic or a cosmetic item.

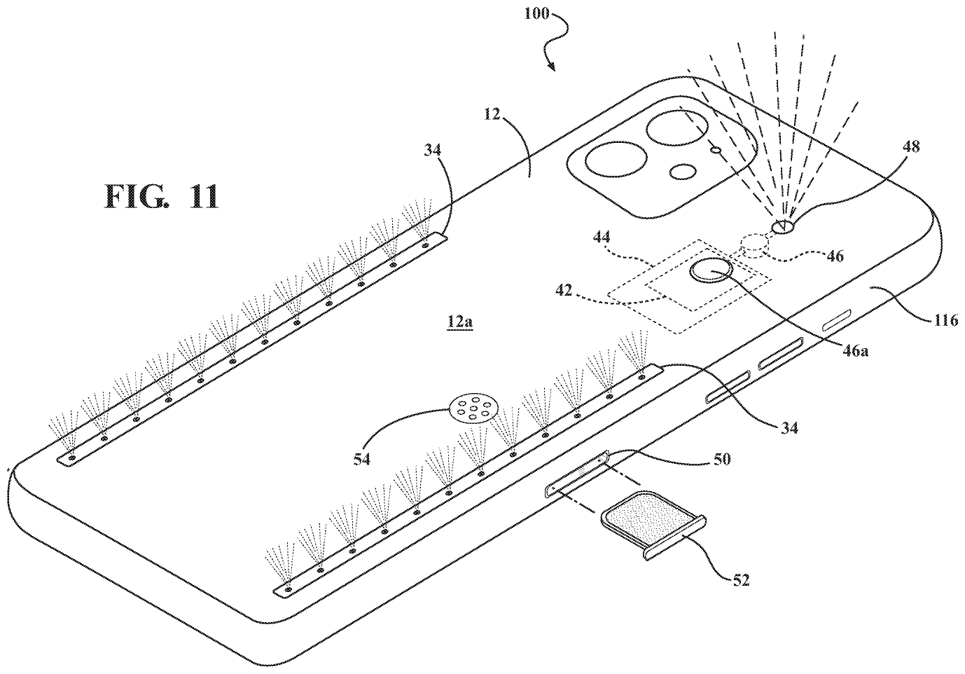

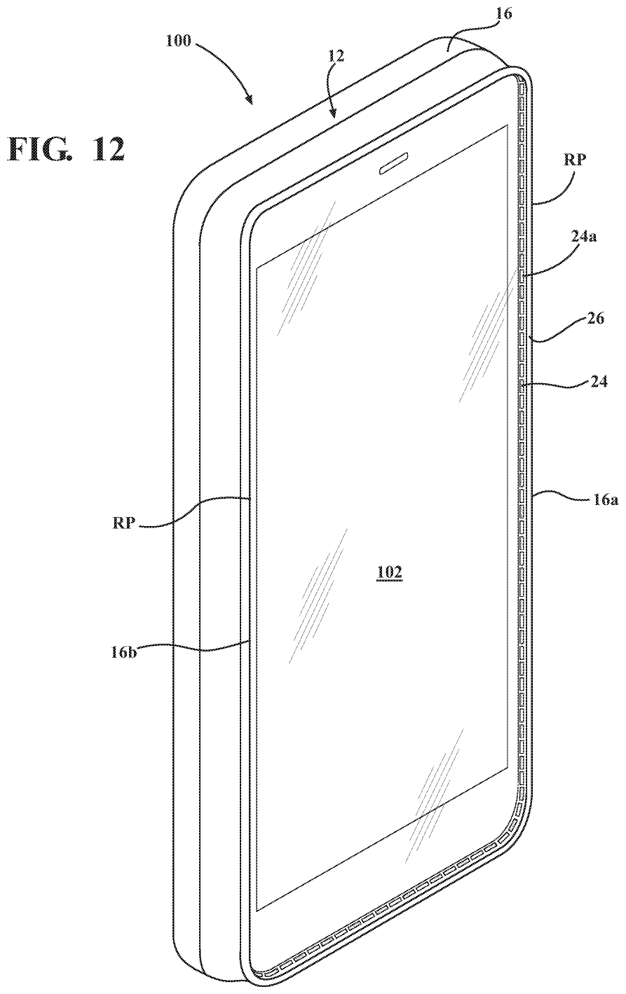

With reference now to FIGS. 11 and 12, a mobile device 100 is provided. The mobile device 100 is configured to perform telecommunications functions, to include web browsing, email, text, phone calls, messaging, and the like. It should be appreciated that the mobile device includes electronic components such as hardware memory, circuits and the like to execute commands for running software applications and the like. The mobile device 100 includes a housing 12 bounding a space 100a configured to hold electronic components for executing telecommunications. The housing 12 includes a back wall 12a opposite of an exposed surface 102, the exposed surface being a surface configured to receive tactile responses for performing telecommunications.

The mobile device 100 further includes a first light emitting device 24 disposed within the housing 12. A light guide 26 is integrally formed to the housing 12. The first light emitting device 24 is optically connected to the light guide 26. The light guide 26 is configured to direct light over the exposed surface 102 of the mobile device 100. FIG. 12 shows an aspect, where the light guide 26 is disposed along at least a side of the housing 12. Preferably, the first light emitting device 24 is configured to emit ultraviolet light. The light guide 26 is shown as having a reflective point “RP” which reflects the light through a side wall 26a of the light guide 26, shining ultraviolet light onto the exposed surface of the mobile device 100. The reflective point RP is disposed above the exposed surface 102.

In yet another aspect of the mobile device 100 wherein the light guide 26 is disposed along a respective first peripheral edge 16a and a second peripheral edge 16b of the housing 12. The second peripheral edge 16b being opposite of the first peripheral edge 16a. A pair of first light emitting devices 24 may be optically coupled to the light guide 26 so as to be disposed along a respective first peripheral edge 16a and second peripheral edge 16b of the housing 12. In yet another aspect of the mobile device 100, the light guide 26 bounds the entire periphery of the housing 12.

With reference now to FIG. 11, the mobile device may further include a second emitting device 34. The second light emitting device 34 is disposed on a back surface of the housing 12 so as to be opposite of the open top 18. The second light emitting device 34 may be fixed or removably attached to the back surface of the housing 12. In an aspect where the second light emitting device 34 is removably attached. The mobile device 100 includes a light device housing 36 disposed on a back surface of the base 14. The light device housing 36 is configured to fittingly receive the second light emitting device 34. Preferably, the light device housing 36 has a depth configured to retain the outer surface of the second light emitting device 34 flush with the back surface of the base 14 of the housing 12.

The mobile device 100 may further include a wire 38 electrically coupling the second light emitting device 34 to the battery 28 so as to power the second light emitting device 34 when the second light emitting device 34 is removed from the housing 12. The mobile device 100 may include a spooling mechanism 40 configured to spool the wire 38 so as to allow the second light emitting device 34 to extend from the mobile device 100 and to retract and spool the wire 38 when the second light emitting device 34 is seated in the light device housing 36. In such an aspect, the second light emitting device 34 may be used as a wand to disinfect various surfaces. The concept, of a removably attached second light emitting device 34 for use in a mobile device 100 is illustratively shown in FIG. 5.

The mobile device 100 may include a first cartridge 42 configured to hold a fluid. In particular, the first cartridge 42 is configured to hold an antiseptic. The housing 12 includes a compartment 44 configured to receive the first cartridge 42. The mobile device 100 further includes a dispensing mechanism 46 fluidly coupled to the first cartridge 42 when the first cartridge 42 is disposed within the compartment 44 so as to dispense the fluid through a port 48 disposed on a back wall 12a of the housing 12. For illustrative purposes, the dispensing mechanism 46 may be a pump which is actuated by pressing a button 46a disposed on a back of the base 14.

The mobile device 100 may be further configured to emit a fragrance. The mobile device 100 includes a slot 50 disposed along the peripheral wall 16 of the housing 12. The mobile device 100 further includes a second cartridge 52 configured to emit a scent and at least one hole 54 disposed on the back wall 12a of the housing 12, the at least one hole 54 in fluid communication with the slot 50. Accordingly, the second cartridge 52 is seated within the slot 50 and the hole 54 is preferably located above the second cartridge 52 so as to allow fragrance from the second cartridge 52 to diffuse freely through the holes 54.

While particular embodiments have been illustrated and described herein, it should be understood that various other changes and modifications may be made without departing from the spirit and scope of the claimed subject matter. Moreover, although various aspects of the claimed subject matter have been described herein, such aspects need not be utilized in combination. It is therefore intended that the appended claims cover all such changes and modifications that are within the scope of the claimed subject matter. Accordingly, the case 10 and the mobile device 100 described herein is not intended to have each of the features that are set forth in the specification, but may be manufactured to be modular based upon consumer desire. For instance, the case 10 and the mobile device 100 may be manufactured with just a first light emitting device 24, or just a second light emitting device 34, or just a first cartridge 42 or just a second cartridge 52 or a combination of any of these features.

What is claimed is:

1. A case for a mobile device, the case comprising:
a housing having an open top, the housing configured to receive the mobile device;
a first light emitting device disposed within the housing; and
a light guide, the first light emitting device optically connected to the light guide, the light guide configured to direct light over an exposed surface of the mobile device.

2. The case as set forth in claim 1, further including a battery configured to power the first light emitting device.

3. The case as set forth in claim 1, wherein the first light emitting device is configured to emit ultraviolet light.

4. The case as set forth in claim 1, wherein the first light emitting device is integrally formed to the housing.

5. The case as set forth in claim 1, wherein the light guide is disposed along a first peripheral edge of the housing.

6. The case as set forth in claim 5, wherein the light guide is a pair of light guides, wherein one of the pair of light guides is disposed along the first peripheral edge of the housing and the other of the pair of light guides is disposed along a second peripheral edge of the housing, the second peripheral edge being opposite of the first peripheral edge.

7. The case as set forth in claim 1, further including a second light emitting device, the second light emitting device disposed on a back surface of the housing, the back surface opposite of the open top.

8. The case as set forth in claim 1, further including an actuator configured to actuate the first light emitting device.

9. The case as set forth in claim 1, wherein the first light emitting device is also configured to emit visible light.

10. A case for a mobile device, the case comprising:
a housing having an open top, the housing configured to receive the mobile device; and
a second light emitting device disposed on a back surface of the housing, the back surface opposite of the open top.

11. The case as set forth in claim 10, further including a battery configured to power the second light emitting device.

12. The case as set forth in claim 11, wherein the second light emitting device is configured to emit ultraviolet light.

13. The case as set forth in claim 12, wherein the second light emitting device is removably attached to the back surface of the housing.

14. The case as set forth in claim 13, further including a wire electrically coupling the second light emitting device to the battery so as to power the second light emitting device when the second light emitting device is removed from the housing.

15. A case for a mobile device, the case comprising:
a housing having an open top, the housing configured to receive the mobile device;
a first cartridge configured to hold a fluid;
a compartment disposed within the housing, the compartment configured to receive the cartridge; and
a dispensing mechanism fluidly coupled to the first cartridge when the first cartridge is disposed within the compartment so as to dispense the fluid through a port disposed on a back surface of the housing.

16. The case as set forth in claim 15, further including a slot disposed along a peripheral side of the housing and a second cartridge configured to emit a scent and at least one hole disposed on the back surface of the housing, the at least one hole in fluid communication with the slot.

17. The case as set forth in claim 15, further including a first light emitting device disposed within the housing; and
a light guide, the first light emitting device optically connected to the light guide, the light guide configured to direct light over an exposed surface of the mobile device.

18. The case as set forth in claim 17, wherein the first light emitting device is configured to emit ultraviolet light.

19. The case as set forth in claim 18, wherein the first light emitting device is integrally formed to the housing.

20. The case as set forth in claim 19, wherein the light guide is disposed along a first peripheral edge of the housing.

21. The case as set forth in claim 20, wherein the light guide is disposed along a first peripheral edge of the housing and a second peripheral edge of the housing, the second peripheral edge being opposite of the first peripheral edge.

22. The case as set forth in claim 21, further including a second light guide, the second light guide disposed on a back surface of the housing, the back surface opposite of the open top.

23. The case as set forth in claim 21, further including an actuator configured to actuate the first light emitting device.

24. The case as set forth in claim 23, wherein the actuator is configured to actuate the first light emitting device for a predetermined period of time.

25. The case as set forth in claim 24, wherein the first light emitting device is also configured to emit visible light.

26. A case for a mobile device, the case comprising:
a housing having an open top, the housing configured to receive the mobile device; and
a first cartridge removably attached to a back surface of the case.

27. The case as set forth in claim 26, wherein the case includes a groove disposed on an exterior surface of a base of the housing, and wherein the first cartridge includes a tab configured to slide within the groove so as to secure the first cartridge to the base.

28. The case as set forth in claim 27, wherein the first cartridge includes a dispensing mechanism configured to dispense a fluid.

29. The case as set forth in claim 28, wherein the fluid is one of either an antiseptic or an air freshener.

30. The case as set forth in claim 27, wherein the first cartridge includes a cover configured to open or close a space so as to provide access or contain an item stored within the space.

31. The case as set forth in claim 30, wherein the item is a cosmetic item.

32. A mobile device configured to perform telecommunications, the mobile device comprising:
a housing bounding a space configured to hold electronic components for executing telecommunications functions, the housing including a back wall opposite of an exposed surface, the exposed surface being a surface configured to receive tactile responses for performing telecommunications functions;
a first light emitting device disposed within the housing; and
a light guide, the first light emitting device optically connected to the light guide, the light guide configured to direct light over the exposed surface of the mobile device.

33. The mobile device as set forth in claim 32, wherein the light guide is disposed along at least a side of the housing.

34. The mobile device as set forth in claim 33, wherein the first light emitting device is configured to emit ultraviolet light.

35. The mobile device as set forth in claim 34, wherein the light guide is disposed along a first peripheral edge and a second peripheral edge of the housing, the second peripheral edge being opposite of the first peripheral edge.

36. The mobile device as set forth in claim 35, further including a second emitting device, the second emitting device disposed on a back wall of the housing.

37. The mobile device as set forth in claim 36, a first cartridge configured to hold a fluid;
a compartment disposed within the housing, the compartment configured to receive the cartridge; and
a dispensing mechanism fluidly coupled to the first cartridge when the first cartridge is disposed within the compartment so as to dispense the fluid through a port disposed on a back surface of the housing.

38. The mobile device as set forth in claim 37, further including a slot disposed along a peripheral side of the housing and a second cartridge configured to emit a scent and at least one hole disposed on the back wall of the housing, the at least one hole in fluid communication with the slot.
